(19) United States
(12) Patent Application Publication
(10) Pub. No.: US 2002/0029015 A1
(43) Pub. Date: Mar. 7, 2002

(54) Device for administering a composition in a duct of a human or animal body

(76) Inventors: Edoardo Camenzind, Geneva (CH); Pascal Neuville, Strasbourg (FR); Lionel Fontao, Annemasse (FR); Valerie Calenda, Strasbourg (FR)

Correspondence Address: Norman H. Stepno, Esquire, BURNS, DOANE, SWECKER & MATHIS, L.L.P., P.O. Box 1404, Alexandria, VA, 22313-1404, US

(21) Appl. No.: 09/897,476

(22) Filed: Jul. 3, 2001

(30) Foreign Application Priority Data
Jul. 4, 2000 (FR) 00 08751
Mar. 9, 2001 (FR) 01 03286

(51) Int. Cl. A61M 29/00

(52) U.S. Cl. 604/97.02

(57) ABSTRACT

Device (2) for administering a composition in a duct (14) of a human or animal body, comprising means (4) able to enter an inner surface of the duct wall to make blind openings in a thickness of the wall and dispenser means (20) to place the composition in contact with the openings.

The invention concerns a device and method for use in the administration of a composition in a duct wall of a human or animal body, especially for the treatment or prevention of atherosclerosis, in particular to combat restenosis subsequent to the implantation of a stent in a blood vessel, an artery in particular.

Atherosclerosis (Ross, 1999, Am. Heart. J. 138, 419-20) is a disease of the arteries characterized by invasion of the intima by several cell populations (smooth muscle cells forming the wall of the vessel and inflammatory cells) and build-up of collagen substances and calcium leading to increasing stiffness of the vascular wall and narrowing of the artery lumen. One of the most serious consequences of the blocking of the vessels, also called stenosis, affects the coronary arteries whose role is to irrigate the heart. Called coronary deficiency this disorder causes myocardial ischaemia whose most frequently associated syndrome is myocardial infarction (Roberts, 1998, Am. J. Cardiol 82, 41T-44T).

Two forms of treatment for atherosclerosis-induced stenosis are currently available to patients.

The first type of treatment, called coronary bypass, is chosen when arterial stenosis is major and multiple (Eagle et al., 1999, J. Am. Coll. Cardiol. 34, 1262-347). It is surgical treatment which aims at restoring blood flow to the myocardium by by-passing the blocked coronary artery. To achieve this, a section of mammary artery or saphenous vein is grafted to above and below the stenosed part. This heavy procedure requiring the opening of the chest cavity is only performed in a limited number of cases when the second form of surgical treatment proves inapplicable.

The second approach, called percutaneous transluminal coronary angioplasty consists, during a first step, of inserting a catheter inside the coronary artery at the site of blockage, one end of the catheter being fitted with a balloon. The second step in the procedure is to inflate the balloon in situ so as to compress the atheromatous plaque against the vessel wall restoring sufficient coronary opening to allow satisfactory myocardial irrigation (Cishek and Gershony, 1996, Am. Heart. J. 131, 1012-7). This second technique is the one most frequently used in patients suffering from coronary deficiency It accounts for 50000 surgical operations in France per year and 500000 per year in the United States. However the trauma suffered by the atheromatous artery during dilatation of the balloon, in 30% of cases, leads to the onset of a new lesion, called restenosis at the site of dilatation (Hong et al., 1997, Curr Probl Cardiol, 22, 1-36). This restenosis characterized by further narrowing of the artery is in fact due to the onset of two successive phenomena. Firstly arterial remodelling occurs which is a constriction of the vessel in response to the dilatation phenomenon and occurs in acute manner during the hours following after the procedure (Pasterkamp et al., 2000, Cardiovasc Res. 45, 843-52). Secondly restenosis may be caused by excessive scar healing characterized by a proliferation of smooth muscle cells (SMC) and abundant synthesis of extracellular matrix (ECM) leading to symptomatic re-obstruction of the treated coronary artery in the months following after the angioplasty (Schwartz et al., 1996, Int. J. Cardiol. 53, 71-80).

This remodelling phenomenon can he overcome by use of the <<stenting>> technique performed after angioplasty which consists of inserting a reinforcement, generally a metal mesh tube called a stent. The stent fits to the contour of the vessel wall imparting artificial mechanical rigidity to the artery, which prevents the occurrence of the acute constriction phase and provides a wider arterial diameter. Within a few years this procedure has been given general use and is henceforth standard procedure in cardiology surgery (Goy and Eeckout, 1998, Lancet 351, 1943-9).

However, although this technique has brought a notable improvement in the short-term prognosis of patients treated by angioplasty, recurrent blockage or restenosis still occurs in 30 to 50% of patients within six months after the implantation of the stent.

It is nonetheless important to note that in such cases the arterial narrowing at the site of the stent is solely related to cell proliferation and does not involve the phenomenon of arterial remodelling. In this case the term intra-stent restenosis is used which is currently treated by re-dilatation of the obstructed area by means of repeat angioplasty. Unfortunately, this treatment leads to more frequent and more rapid re-restenosis of the dilated lesion. (Rossi et al., 2000, J. Am. Coll. Cardiol. 35, 1569-76).

The high incidence of the phenomenon of restenosis in patients treated by angioplasty and/or stent implantation raises a veritable public health problem responsible for an estimated cost of 2 billion dollars per year in the United States. To date, no effective pharmacological treatment is available for the prevention of restenosis whether related to angioplasty and/or stent implantation.

Brachytherapy based on positioning a catheter fitted with a radioactive source at the site of arterial narrowing can overcome cell hyperplasia (Waksman et al., 2000, Circulation 101, 2165-71). However, this procedure which leaves the wall unhealed, leads to late thrombosis and is accompanied by cell proliferation at the margins of the irradiated vascular segment (Waksman, 1999, Circulation 100, 780-2). At the current time, it does not offer satisfactory treatment.

Another approach currently being evaluated concerns the development of gene therapy. Gene therapy can be fairly broadly defined as the transfer of genetic information of interest to a cell or host body. Most gene therapy strategies use transfer vectors to convey this information to and into the cell target. Numerous transfer vectors, whether viral, synthetic or plasmid, have been developed in recent years and have been the subject of numerous publications accessible to persons skilled in tho art (see for example Robbins et al., 1988, Tibtech, 16, 34-40 and Rolland, 1998, Therapeutic Drug Carrier systems, 15, 143-198).

Moreover, extensive experimental data is available concerning the transfer of such vectors containing genetic information of interest, genes in particular, into arterial cells. By way of example, adencoviral vectors may be cited which make it possible to consider a gene approach for the prevention and/or treatment of restenosis. For example the transfer of genes encoding inhibitors of the migration and proliferation of smooth muscle cells of the arterial wall appears to open up a promising path for treatment (Kibbe et al., 2000, Circ. Rebi. 86, 829-33; Macejak et al., 1999, J. Virol. 73, 7745-51; Claudio et al., 1999, Circ. Res. 85, 1032-9; Perlman et al., 1999, Gene Ther. 6, 758-63). However, several problems remain to be solved before intravascular gene therapy becomes part of clinical practice, especially problems related to the efficacy of vector transfer into the arteries.

Indeed, the transfer of vectors to the normal or atheromatous vascular wall, in particular to its constituent cells, remains limited in efficacy. The elastic laminae which impart plasticity to the arteries form a barrier hindering the deep penetration of the transfer vectors, and the presence of calcified atheromatous plaque in patients further reduces the efficacy of this transfer (Maillard et al., 1998, Gene Ther. 5, 1023-30; Rekhter et al., 1998, Circ. Res. 82, 1243-1252). Similarly, the restenotic tissue formed for the most part of smooth muscle cells and inflammatory cells contains an abundant extracellular matrix which forms a barrier considerably reducing the transfer of the vectors to and into the target cells.

In addition, the intra-coronary administration of transfer vectors is made difficult by the heart's oxygenating function carried out by these arteries. Reported experiments in genie therapy conducted on the carotid or femoral arteries of rats or rabbits require the obstruction of blood flow in order to contact the composition containing said transfer vector with the vascular wall for sufficient time to produce maximum administration efficacy and hence transfer of the vector to the cells. This approach is not compatible with the function of the coronary arteries for it is impossible to obstruct the flow in these vessels for a long time without causing a serious cardiac disorder due to insufficient oxygenation. Consequently, the contact time between the coronary arterial cells and the composition containing the transfer vector must necessarily be very short which often leads to low vector transfer efficacy to and into the target cells of the treated vessel wall.

In this context, one purpose of the invention is to provide a method and device with which it is possible to administer a composition quickly and efficiently in the wall of a duct of a human or animal body, even if a fluid may circulate within this duct. More particularly, one purpose of the invention is to provide the possibility of administering transfer vectors, or compositions containing the same, quickly and as efficiently as possible to target cells located in particular in the thickness of the wall of said duct. More especially, this efficient administration of said vector or said composition leads to efficient transfer of said vector to or/and into said cells.

To achieve this purpose, the invention firstly concerns a method for administering a composition in a wall of a human or animal duct, characterized in that it comprises the steps consisting of:

entering an inner surface of the duct wall to make blind openings in a thickness of the wall; and

placing the composition in contact with the openings made in the wall.

The method may be conducted using two separate devices (example 1) each one used to carry out either one of these steps, or using a single device combining the two properties (examples 2 and 3), that is to say a single device to carry out the two above-mentioned steps.

According to one particular embodiment, the invention concerns such a method for the treatment or prevention of restenosis or re-restenosis, and more particularly when it occurs at the site of the stent. In one particular case of the invention, a said stent has been placed in said human or animal duct after treatment of said duct by angioplasty. According to one particular embodiment, said composition preferably contains at lest one transfer vector. According to another embodiment of the invention, said composition contains a drug able to treat or prevent said restenosis or said re-restenosis.

Therefore, by means of the openings in the thickness of the wall, the composition is placed in direct contact with the wall cells, and more particularly with the cells which are located in the space between the neointima and the elastic lamina. The administration of this composition, or of the compounds contained in said composition, is therefore effective, even if the contact time is short, for example if a fluid is circulating within the duct.

In the particular case of administration using the transfer vector means of the invention, or of a composition containing said vector, to combat resteriosis or re-restenosis of an artery, it was experimentally found that the accessibility of the vectors to the wall cells and their transfer into the cells was more generalized and extended deeper into the thickness of this wall, making it possible to envisage, over the longer term, more efficient prevention of restenosis or re-restenosis.

Under the present invention, by <<to enter >> is meant to indicate that cuts and/or perforations and/or erosions are made in the thickness of the duct wall to make openings. <<To cut>>, <<to shear>>, <<to slice>>, <<to incise>> or <<to section>> and <<to pierce>> <<to punch>> or <<to bore>> or <<to erode>> or <<to fray>> are synonyms of <<enter>> within the scope of the present invention. The <<openings>> in the meaning of the invention are called <<blind>> since they are not perforated end to end in the duct wall. These openings have different appearances with no particular limitation as to their section or orientation. Therefore said openings may have the appearance of a cut of variable width (for example 0.5 to 10 mm, preferably 2.5 to 5 mm) as may be obtained for example with a blade, razor or knife. Such openings may also have the appearance of a hole, a pinprick of variable diameter (for example from 0.05 to 1 mm) as may be obtained for example with a sharp tip, punch, trocar. Said openings may also have the appearance of thinning or of a rubbed surface such as may be obtained with a scraper, a rough surface, abrasive, for example. Said openings may also have a diffuse, necrosed appearance as may be obtained for example through the action of a chemical compound, appropriate localized radiation. The openings obtained with the invention may be made longitudinally or transversely relative to the axis of the duct; also they may be invariably made along a radial or oblique axis relative to the thickness of said duct.

The parts of the device according to the invention used to obtain said openings may be made of different materials such as for example a metal or an alloy, e.g. a cobalt, nickel and/or titanium based alloy, some stainless steels; a polymer containing polypropylene for example, PEEK, HDPE (high density polyethylene), polysulfone, acetyl, PTRE, PEP, polycarbonate urethane, polyurethane, silicon, PTFE, ePTFE or polyolefin. They may also be made of a biologically acceptable material.

The method of the invention may also have any one of the following characteristics:

the inner surface is entered by making incisions in the wall;

the incisions are made in a radial direction relative to a longitudinal direction of the duct;

prior to the step consisting of entering the inner surface, the area to be entered is dilated;

the openings are placed in contact with the composition by causing the composition to circulate in channels of which one surface is formed by the inner surface of the duct;

the openings are placed in contact with the composition by causing the composition to circulate in the channels of which one surface is formed by a wall having outer openings;

the duct is a blood vessel, an artery for example;

the vessel is partially obstructed;

the vessel is fitted with a stent;

the composition is intended to implement treatment by gene therapy;

Finally, according to the invention a method is provided to administer a composition in a wall of a duct in a human or animal body, characterized in that it comprises the steps consisting of:

inserting the device of the invention in the duct;

radically extending the cutting or perforation parts to enter the inner surface of the wall by making blind openings in the thickness of the wall;

arranging dispenser means;

radially extending the dispenser means; and

placing the composition in contact with the openings.

The invention also concerns a device for administering a composition in a wall of a duct of a human or animal body, with device comprising means able to enter an inner surface of the wall of the duct to make blind openings in a thickness of the wall, and comprising dispenser means to place the composition in contact with the openings.

In addition, the device may offer at least one of the following characteristics:

the entry means comprises cutting or perforating parts;

the entry means are extensible in radial direction relative to an axial direction of the device;

the entry means are associated with an inflatable chamber;

the entry means are carried by a wall of the inflatable chamber;

the entry means are associated with a tube on which a inflatable chamber is mounted;

the entry means are cutting or perforating parts;

the cutting or perforating means are carried by the tube in which the inflatable chamber is mounted,

the entry means comprise arms carrying the cutting or perforating parts;

the arms surround the inflatable chamber;

the dispenser means are racially extensible relative to an axial direction of tho device;

the dispenser means have channels able to receive the composition, the channels being open in opposite direction to an axis of the device;

the dispenser means comprise a wall provided with outer openings;

the dispenser means are able to surround the entry means;

the dispenser means are able to slide relative to the entry means in an axial direction of the device;

the inflatable chamber is able to extend the dispenser means radially;

the device is intended to administer a composition in the wall of a blood vessel such as an artery, in particular an artery fitted with a stent;

it is a catheter.

In addition, the invention provides for a device for administering a composition in a wall of a human or animal duct, the device comprising means able to enter an inner surface of the wall of the duct to make blind openings in the thickness of this wall, these means carrying cutting or perforating parts and being radially extensible relative to an axis of the device, the device comprising dispenser means to place the composition in contact with the openings, the dispenser means being extensible radially and able to surround the entry means. The entry means of the invention are such that they can be used to make blind openings in a thickness of the wall as described previously.

The method and device of the invention concern the in vivo administration of compositions, pharmaceutical compositions in particular.

According to one preferred embodiment, these compositions are intended for the implementation of gene therapy treatment. In this case, said composition contains at least one genetic data item of interest, preferably associated with a transfer vector which is intended to enable or facilitate the transfer of this information to and/or into the target cells. Said genetic data item of interest consists of, or is included in, a nucleic acid sequence.

By <<nucleic acid>> or “nucleic acid sequence” is meant a DNA and/or RNA fragment, double strand or single strand, linear or circular, natural isolated or synthesized, designating a precise chain sequence of nucleotides, whether modified or not, making it possible to define a fragment or a region of a nucleic acid with no limitation as to size. According to one preferred embodiment, this nucleic acid is chosen from the group consisting of a cDNA; a genomic DNA; plasmid DNA; a messenger RNA; an antisense RNA; a ribozyme; a transfer RNA; a ribosomic RNA; or a DNA coding for such RNAS. In best preferred manner, said nucleic acid codes for a polypeptide; in this case the term gene is used.

A <<transfer vertor>> according to the invention is intended to enable or facilitate the transfer of said genetic information or/and of said nucleic acid to or/and into the target cells. It may for example be a plasmid free of any compound facilitating its insertion into the cells but comprising said genetic information; a said plasmid or a said nucleic acid containing said genetic information associated with at least one polypeptide, in particular a polypeptide of viral origin, and more particularly of adenoviral or retroviral origin, preferably a said nucleic acid incorporated in an infectious viral particle (in one preferred case said nucleic acid consists of a viral genome that is optionally modified as proposed below and recombined in the sense that it cantinas said genetic information of interest), or a synthetic polypeptide; a nucleic acid associated with a ligand.

In preferred manner according to the present invention, <<transfer vector>> designates a recombinant vector of plasmid or viral origin. The choice of plasmids which may be used within the scope of this invention is vast. They may be cloning and/or expression vectors. In general, they are known to persons skilled in the art and many of them are commercially available, but it is also possible to build or modify them using genetic engineering techniques. As examples mention may be made of plasmids derived from pBR322 (Gibco BRL), pUC (Gibco BRL), pBluescript (Stratagene), pREP4, pCEP4 (Invitrogene) or further p Poly (Lathe et al., 1987, Gene 57, 193-201). Preferably, a plasmid used under the present invention contains a replication origin ensuring initiation of replication in a producer cell and/or host cell (for example, the ColE1 origin will be chosen for a plasmid intended to be produced in E. coli and the oriP/EBNA1 system if it is desired to be self-replicating in a host mammalian cell (Lupton et Levine, 1985, Mol. Cell. Biol. 5, 2533-2542; Yates et al., Nature 313, 812-815) It may also contain a selection gene with which to select or identify the transfected cells (for example complementation of an auxotrophy mutation, a gene encoding resistance Lo an antibiotic). Evidently, it may comprise additional elements improving its maintaining and/or stability within a given cell (cer sequence which promotes maintaining in plasmid monomer form (Summers and Sherrat, 1984, Cell 36, 1097-1103), integration sequences in the cell genome.

In the case of a viral vector, it is possible to consider a vector derived from a poxvirus (virus of the vaccine for example, in particular MVA, canaripox), from an adenovirus, a retrovirus, a herpes virus, an alphavirus (for example virus of the Togavirus family, especially Semliki Forest virus), a foamy virus or from a virus associated with the adenovirus. Preferably recourse is made to a non-replicating and non-integrating vector. In this respect, the adenoviral vectors are particularly suitable for the implementation of the present invention. However, it should be noted that for the application of the present invention the type of vector is of little importance.

Retroviruses have the property of infecting and majority integrating into the dividing cells, and in this respect they are particularly suitable for the application which aims at acting on the phenomenon of restenosis. One recombinant retrovirus which may be used within the scope of the invention generally comprises the LTR sequences, an encapsulation region and tho nucleotide sequence of the invention placed under the control of the retroviral LTR or an internal promoter such as those described below. It may be derived from a retrovirus of any origin (murine, primate, feline, human, etc.) and in particular from MoMuLV (Moloney murine leukemia virus), MVS (Murine sarcoma virus) ou Friend murine retrovirus (Fb29). It is propagated in an encapsulation line able to provide en trans supply of the viral polypeptides gag, pol and/or env required for forming a viral particle. Such lines are described in the literature (PA317, Psi CRIP GP+Am-12 etc.). The retroviral vector of the invention may comprise modifications in particular at the LTRs (replacement of the promoter region by an eukaryote promoter) or of the encapsulation region (replacement by a heterologous encapsulation region, for example of typo VL30) (see French applications FR 94 08300 and FR 97 05203).

It is also possible to have recourse to a defective adenoviral vector for replication, that is to say devoid of all or part of at least one region essential for replication chosen from among the regions E1, E2, E4 and/or L1-L5. Deletion of the E1 region is preferred. But it may be combined with other modification(s)/deletion(s) in particular affecting all or part of the regions E2, E4 and/or L1-L5, insofar as the essential defective functions are complemented en trans by means of a complementation line and/or an auxiliary virus in order to ensure the production of the viral particles of interest. In this respect, recourse may be made to vectors of the prior art, such as for example those described in international applications WO 94/28152 and WO 97/04129. By way of illustration, the deletion of the majority part of the E1 region and of the E4 transcription unit is particularly advantageous. For the purpose of increasing cloning capacity, the adenoviral vector may also be deprived of all or part of the non-essential E3 region. According to another alternative, a minimal adenoviral vector may be used which only withholds the sequences essential for encapsulation, namely the 5′ and 3′ ITRs (Inverted Terminal Repeat) and the encapsulation region. Moreover, the origin of the adenoviral vector of the invention may be varied both in respect of species and of serotype. It may be derived from the genome of an adenovirus of human or animal origin (for example canine, avian, bovine, murine, ovine, porcine, simian) or from a hybrid comprising fragments of the adenoviral genome of at least two different origirib. Particular mention nay be made of the adenoviruses CAV-1 or CAV-2 of canine origin, DAV of avian origin or even type 3 Bad of bovine origin (Zakharchuk et al., Arch. Virol., 1993, 128: 171-176; Spibey et Cavanagh, J. Gen. Virol., 1989, 70: 165-172; Jouvenne et al., Gene, 1987, 60: 21-28; Mittal et al., J. Gen. Virol., 1995, 76: 93-102). However, preference is given to an adenoviral vector of human origin preferably derived from an adenovirus of C serotype, in particular type 2 or 5. One adenoviral vector of the invention may be generated in vitro in Esclerichia coli (E. coli) by ligation or homologous recombination (see for example WO 96/17070) or by recombination in a complementation line. The different adenoviral vectors and their preparation techniques are known (see for example Graham and Prevect, 1991, in Methods in Molecular Biology, vol 7, p 109-128; Ed: E. J. Murey, The Human Press Inc).

It is also possible for replication to have recourse to a replicating or conditionally defective viral vector. Such vectors are well known to persons skille din the art and are abundantly described in the literature.

If a non-viral vector is concerned, it will more specifically relate to the case in which a plasmid vector such as presented above is associated with a compound or a combination of several compounds facilitating its transfer to inside the cells. With such compounds it is possible in particular to improve transfection efficacy and/or the stability of a vector, especially a vector of plasmid origin, and/or the protection of said vector in vivo against the immunity system of the host body (Rolland A, Critical reviews in Therapeutic Drug Carrier System, 15, (1998), 143-198). These substances associate themselves with the nucleic acids by electrostatic, hydrophobic, cationic, covalent or preferably non-covalent interaction. Such substances are widely documented in the literature accessible to persons skilled in the art (see for example Felgner et al., 1987, Proc. West. Pharmacol. Soc. 32, 115-121; Hodgson and Solaiman, 1996, Nature Biotechnology 14, 339-342; Remy et al., 1994, Bioconjugate Chemistry 5, 647-654). By way of non-restrictive illustration, they may be cationic polymers, cationic-lipids, but they may also be liposomes, nuclear or viral proteins or even neutral lipids. These substances may be used alone or in combination. Examples of such compounds, and of the methods which may be used to measure their capacity for improving transfection efficacy and/or the stability of a given vector, are given in particular in patent applications WO 98/08489, WO 96/17693, WO 98/34910, WO 98/37916, WO 98/53853, EP 890362 or WO 99/05183. They may in particular be lipid substances such as DOTMA (Felgner et al., 1987, PNAS, 84, 7413-7417), DOGS or Transfectam™ (Behr et al., 1989, PNAS, 86, 6982-6986), DMRIE or DORIE (Felgner et al., 1993, Methods, 5, 67-75), DC-CHOL (Gao et Huang, 1991, BBRC, 179, 280-285), DOTAP™ (McLachlan et al., 1995, Gene Therapy, 2, 674-622) or Lipofectamine™. The compound may also be a cationic polymer such as polyamidoamine for example (Haensler and Szoka, Bioconjugate Chem. 4 (1993), 372-379), a “dendrimer” polymer (WO 95/24221), an imine polyethylene or imine polypropylene (WO 96/02655), chitosan, a poly(aminoacide) such as polylysine (U.S. Pat. No. 5,595,897 or FR-2 719 316); a polyquaternary compound; protamine; polyimines; imine polyethylene or imine polypropylene (WO 96/02655); polyvinylamines; DEAE substituted polycationic polymers, such as the pullulanes, celluloses; polyvinylpyridine; polymethacrylates; polyacrylates; polyoxethanes; polythiodiethylaminomethylethylene (P(TDAE)); polyhistidine; polyornithine; poly-p-aminostyrene; polyoxethanes; co-polymethacrylates (for example HPMA copolymers; N-(2-hydroxypropyl)-methacrylamide); the compounds described in U.S. Pat. No. 3,910,862, DEAE polyvinylpyrrolide complexes with methacrylate, dextran, acrylamide, polyimines, albumine, 1-dimethylaminomethylmethacrylate and the ammonium chloride of polyvinylpyrrolidonemethylacrylaminopropyltrimethyl; the polyamidoamines; telomeric compounds (patent application EP 98401471.2). Nevertheless this list is not exhaustive and other known cationic polymers may be used to obtain the nucleic acid complexes of the invention. In addition, these lipids and cationic polymers may be fluorinated (see for example WO 98/34910). In one advantageous case, such non-viral vectors also contain an adjuvant for example a neutral, zwitterionic or negatively charged lipid. These neutral, zwitterionic or negatively charged lipids may for example be chosen from the group comprising natural phospholipids of animal or plant origin, such as phosphatidylcholine, phosphocholine, phosphatidylethanolamine, sphingomyeline, phosphatidylserine, phosphatidylinositol, ceramide or cerebroside, and their analogues; the synthetic phospholipids which generally contain, but not exclusively, two identical fatty acid chains such as dimyristoylphosphatidylcholine, dioleoylphosphatidylcholine, dipalmitoylphosphatidylcholine, distearoylphosphatidylcholine, phosphatidylethanolamine (PE) and phosphatidylglycerol, and their analogues; phosphatidylcholine, cardiolipine, phosphatidylethanolamine, mono-, di- or triacylglycerol, and alpha-tocopherol and their analogues; phosphatidylglycerol, phosphatidic acid or the analogue of a similar phospholipid; cholesterol, the glycolipids, fatty acids, sphingolipids, prostaglandines, gangliosides, niosomes, or any other natural or synthetic amphiphile.

According to one preferred case, said genetic information of interest comprises or consists of a <<nucleic acid containing a sequence coding for a polypeptide of interest>> by which it is meant to indicate that said nucleic acid comprises a gene coding for a polypeptide of interest, and expression elements of said gene. The term <<polypeptide>> is meant to be construed without any restriction in respect of size or extent of glycosylation.

Should the nucleic acid contain a sequence coding for a polypeptide of interest, it must be specified that said nucleic acid also comprises the elements needed to ensure the expression of said sequence after transfer to a target cell, in particular promoter sequences and/or regulation sequences effective in said cell, and possibly the sequences required to allow the excretion or expression of the said polypeptide on the on the surface of the target cells. The elements needed for expression are formed of all the elements enabling the transcription of the nucleotide sequence to RNA and the translation of the mRNA to a polypeptide, in particular the promoter sequences and/or regulation sequences effective in said cell, and possibly the sequences required to enable the excretion or expression of the said polypeptide on the surface of the target cells. These elements may be regulative or constituent. The promoter is evidently adapted to the chosen vector and the host cell. By way of example, mention may be made of the eukaryote promoters of the PCK genes (Phospho Glycerate Kinase), MT (metallothioneine; McIvor et al., 1987, Mol. Cell Biol., 7, 838-848), α-1 antitrypsin, CFTR, the promoters of the gene coding for muscle creatin kinase, for actin, for immunoglobulins, for β-actin (Tabin et al., 1982, Mol. Cell Biol., 2, 426-436), SRα (Takebe et al., 1988, Mol. Cell. Biol., 8, 466-472), the early promoter of the SV40 virus (Simian Virus), the LTR of RSV (Rous Sarcoma Virus), the promoter of MPSV, the promoter TK-HSV-1, the early promoter of the CmV virus (Cytomegalovirus), the promoters of the virus of the vaccine p7.5K pH5R, pK1L, p28, p11 and the adenoviral promoters ElA and MLP or a combination of said promoters. The early promoter of Cytomegalovirus (CMV) is given particular preference. It may also be a promoter stimulating the expression of the gene specifically in a smooth muscle cell. The promoters may be cited in particular of the genes of smooth muscle α-actin (Foster et al., 1992, J. Biol. Chem. 267, 11995-12003; Shimizu et al, 1995, J. Biol. Chem 270, 7631-7643), of the myosin heavy chain of smooth muscle (Katoh et al., 1994, J. Biol. Chem. 269, 30538-30545), of dosmin (EPO 999 278; Mericskay et al., 1999, Current Topics in Pathology Vol 93 p7-17 Eds Desinoulière et Tuchweber, Springer-Verlag Berlin Heidelberg), of SM22A (Kim et al., 1997, J. Clin. Invest. 100 1006-14). In respect of specific promoters special consideration may be given to chimeric promoters enabling both strong and specific expression in the smooth muscle cells. For example, a promoter such as described in priority document ES 00 44 10208.7 concerning a chimeric construct containing a specific muscle enhancer and a specific SMC promoter (Smooth Muscle Cell) chosen in particular from among the genes SM α-actin, SM myosin heavy chain (SM-MHC), desmin or SM22α. It is also possible to use a tissue-specific promoter region and/or one which can be activated under certain conditions. The literature provides a large amount of information on such promoter sequences. Also, said nucleic acid may contain at least two sequences, identical or different, having transcriptional promoter activity and/or at least two sequences coding for a polypeptide of interest, identical or different, located in contiguous or distant manner relative to one another, in the same or in reverse direction, provided that the function of transcriptional promoter or the transcription of said sequences is not affected. Similarly, in this type of nucleic acid construct, it is possible to insert <<neutral>> nucleic sequences or introns which are not detrimental to transcription and are spliced before the translation step. Such sequences and their uses are described in the literature (WO 94/29471). Said nucleic acid may also contain sequences required for intracellular transport, for replication and/or integration, for secretion, for transcription or translation. Such sequences are well known to persons skilled in the art. Also, the nucleic acids which may be used under this invention may also be modified nucleic acids so that they are unable to integrate into the genome of the target cell, or nucleic acids stabilized by means of agents such as spermine for example which as such do not have any effect on the efficacy of transfection.

Within the scope of the present invention, it is possible to use the entirety or only part of the nucleic acid sequence coding for the polypeptide of interest, or a derived or muted polypeptide, provided that the function and cytotoxic properties of this polypeptide are preserved. In the meaning of the present invention, by mutation is meant a deletion and/or substitution and/or addition of one or more nucleotides. Also it may be envisaged to use a sequence coding for a hybrid polypeptide derived from fusion of the sequence encoding the polypeptide of interest according to the invention and of the sequence encoding a polypeptide of another type (for example, a cytotoxic, membrane anchoring, secretion polypeptide).

By <<genetic information of interest or nucleic acid sequence coding for a polypeptide of interest or gene>> for application under the invention, in particular for the treatment or prevention of restenosis and/or re-restenosis, it is meant to designate for example genes coding for inhibitors of the migration and proliferation of smooth muscle cells of the artery wall, genes coding for a polypeptide having vasoprotective, cytostatic, proapoptotic or cytotoxic activity. Examples are put forward below or in the following documents whose content form an integral part of the application by reference: Kibbe et al., 2000, Circ. Res. 86, 829-33; Macejak et al., 1999, J. Virol. 73, 7745-51; Claudio et al., 1999, Circ. Res. 85, 1032-9; Perlman et al., 1999, Gene Ther. 6, 758-63.

Examples of polypeptides encoded by the gene of interest according to the present invention, include without limitation:

polypeptides involved in the cell cycle such as p21, p16, the expression product of the retinoblastoma gene (Ab), kinase inhibitors, preferably of cyclin-dependent type GAX, GAS-1, GAS-3, GAS-6, GADD-45 and cyclin A, B et D, inhibitors of c-myc, c-myb, Cdk and H-ras.

polypeptides involved in apoptosis, such as p53, Bas, Bc12, BcllX, Bad or other antagonists,

angiogenic polypeptides such as members of the endothelial growth factor group (VEGF), transforming growth factors TGF and in particular TGFα and β), epithelial growth factors EGF), fibroblast growth factors (FGF, and in particular FGFa and FGFb), tumour necrosis factors (TNF and in particular TNFα and TNFβ), CCN (which includes CTGF, Cyr61, Nov, Elm-1, Cop-1 and Wisp-3), dispersion factors/hepatocyte growth factors (SH/HGF), angiogenin, angiopoietin (in particular 1 and 2), angiotensin-2, cytokines (which include in particular interferons β and γ);

polypeptides able to reduce or inhibit cell proliferation, which include antibodies, toxins, immunotoxins, inhibitor polypeptides, oncogen expressing products (ras, MAP kinase, tyrosine kinase receptors, growth factors), the fas ligand, suicide gene products (for example HSV-tk, cytosine desaminase),

polypeptides able to reduce or inhibit cell migration,

polypeptides able to modulate or regulate the expression of cell genes,

coagulation factors (Factor VIII, Factor IX, . . . ),

enzymes such as urease, rennin, thrombin, metalloproteinases, nitrogen monoxide synthases (eNOS or iNOS), SOD, Catalase, heme oxygenase, the lipoprotein lipase family,

natriuretic peptides A, B and C,

recovery agents of oxidized radicals,

enzyme inhibitors, such as alphalantitrypsine, antithrombin III, the inhibitor of the PAI-1 plasminogen activator, the tissue inhibitor of metalloproteinases (TIMP 1-4),

transcription factors such as nuclear receptors which comprise a DNA binding domain, a ligand binding domain, and a transcription activation or inhibition domain (for example fusion products derived from estrogen, steroid or progesterone receptors,

tracers (β-galactosidase, CAT, luciférase, GFP . . . )

and all polypeptides accepted by the prior art as being helpful in the treatment or prevention of a clinical condition, in particular those for which it is desirable to achieve expression in the cells present in the walls of human or animal ducts, such as vessel wells.

The polypeptide of interest which is coded by the sequence contained in said nucleic acid is preferably chosen from among polypeptides having anti-proliferate or anti-migratory activity, vasoprotective protein factors, angiogenic protein factors and polypeptides having cell apoptosis activation activity, cytokines, proteins encoded by a gene called <<suicide gene>>. Cytokines are molecules naturally produced subsequent to antigenic stimulation or inflammatory reaction (Gillis and Williams, 1998, Curr. Opin. Immunol, 10, 501-503) whose use in the treatment of restenosis has been demonstrated in particular by Stephan D (Mol Med. 1997, 3, 593-9). According to this variant of the invention, the polypeptide of interest preferably designates the interferons β and γ which are able to inhibit the proliferation of smooth muscle cells in vitro and in vivo (Stopeck A, 1997, Cell transplantation, 6, 1-8).

According to the invention, the polypeptide of interest may also be a polypeptide having anti-migratory activity. According to this variant, the polypeptide of interest preferably designates an inhibitor of metalloproteinases (TIMP 1-4) able to inhibit the digestion of the extracellular matrix and therefore able to reduce the migration of smooth muscle cells from the media to the intima (Cheng L. 1998, Circulation, 98, 2195-2201).

According to another variant of the invention, the polypeptide of interest is a polypeptide having vasoprotective activity. According to this variant of the invention, the polypeptide of interest is preferably a vasorelaxant able to recognize the proliferation of smooth muscle cells and to exert vasoprotective action by inducing accumulation of cGMP (Hikaru U, 1997, Circulation, 96, 2272-2279).

According to another variant of the invention, the polypeptide of interest is a polypeptide having angiogenic activity. The potential roles of the platelet-derived growth factor (PDGF), of throinbospondin, of fibroblast factors (FGFs), of transforming growth factors (TGF and particularly TGFα and β) and of epithelial growth factors on the prevention of restenosis have been discussed (Cerek, 1991, Am. J. Cardiol., 68, 24-33) and the role of the endothelial growth factor (VEGF) has more particularly been shown in vivo through its action on the re-endothelialisation of the injured artery (Asaharan Circulation, 1994, 3291-3302).

According to another variant of the invention, the polypeptide of interest is a polypeptide encoded by a gene called <<suicide gene>>. Numerous suicide gene/prodrug pairs are currently available. Special mention may be made of the pairs (a) thymidine kinase of the type 1 simplex herpes virus (TK HSV-1) and acyclovir or ganciclovir (GCV) and (b) cytosine desaminase (CDase) and 5-fluorocytosine (5FC) having demonstrated the ability to inhibit neointimal proliferation in an animal model (Takeshi 0, 1994, Science, 781-784; Harrell R, 1997, Circulation, 96, 621-627) and the pairs purine nucleoside phosphorylase of Esclerichia coli (E. Coli) and 6-methylpurine deoxyribonucleoside (Sorscher et al., 1994, Gene Therapy 1, 233-238); guanine phosphoribosyl transferase of E. Coli and 6-thioxanthine (Mzoz and Moolten, 1993, Human Gene Therapy 4, 589-595).

According to one advantageous case, the invention the case in which said polypeptide of interest has at least one enzymatic activity chosen from among thymidine kinase activity, purine nucleotide phosphorylase activity, guanine or uraicil or orotate phosphoribosyl transferase activity and cytosine desaminase activity.

Finally, the polypeptide of interest may be a polypeptide having an activity of cell apoptosis activation, and more particularly the Fas ligand which is able to inhibit the formation of neointima (Luo Z, 1999, Circulation, 99, 1776-1779).

The sequence coding for the polypeptides of interest of the invention may easily be obtained by cloning, by PCR or by chemical synthesis using conventional techniques. They may be native genes or derived from the latter by mutation, deletion, substitution and/or addition of one or more nucleotides. Moreover, their sequences are widely reported in the literature which can be consulted by persons skilled in the art.

Advantageously the composition intended to be administered, depending upon the type of vector used, contains:

if the vector is of plasmid origin or a viral vector, from 0.01 to 100 mg DNQA, preferably between 0.05 to 10 mg, and in best preferred manner from 0.5 to 5 mg;

if the vector is of viral origin, between 10⁴ and 10¹⁴ pfu (plaque-forming units) and advantageously between 10⁵ and 10¹³ pfu, and preferably between 10⁶ and 10¹² pfu.

These dosages are given by way of indication, the practitioner evidently being able adapt dosage to needs, patient condition, the disorder to be treated or prevented, the gene, the vector, the promoter used, etc. such determination not involving excessive work. In addition, such adjustments are fully independent from the device of the invention or its in vivo use.

According to another embodiment, the composition administered according to the invention is a composition containing an active compound, other than a transfer vector or genetic information or a nucleic acid such as defined above, which it is desired to administer to a human or animal duct, to duct walls in particular. According to the invention by <<active compound>> it is meant to designate one or more biologically active agents, such as anti-inflammatory agents for example which prevent inflammation, compounds preventing restenosis by limiting tissue proliferation, anti-thrombogenic compounds which inhibit or control the formation of thrombus or thrombolysis, or bioactive compounds which regulate tissue growth and stimulate tissue healing. Such active compounds are for example but not limited to steroids, fibronectin, anti-coagulant compounds, anti-platelet compounds, compounds preventing the growth of smooth muscle cells on the inner surface of vessel walls, heparin or fragments of heparin, aspirin, coumarin, the activator of tissue plasminogen (or TPA), urokinase, hirudin, streptokinase, anti-proliferatives (methotrexate, cisplatin, fluorouracil, adriamycin), antioxidants (ascorbic acid, beta carotene, vitamin E), anti-metabolites, inhibitors of thromboxane, non-steroid and steroid anti-inflammatories, calcium pump blockers, immunoglobulins, antibodies, cytokines, lymphokines, growth factors, prostaglandins, leukotrienes, laminin, elastin, collagen or integrins. According to one particular case, such compounds are encapsulated prior to administration using the device of the invention, for example in liposomes, nanoparticles or pharmacosomes. Such encapsulation techniques are widely described in the literature and reference may be made for example to documents U.S. Pat. No. 5,874,111, U.S. Pat. No. 5,827,531, U.S. Pat. No. 5,773,027 or U.S. Pat. No. 5,770,222 whose content is incorporated herein by reference.

The compositions which may be administered using the device of the invention may also be formulated with a vehicle that is pharmaceutically acceptable. Said vehicle is preferably isotonic, hypotonic or scarcely hypertonic and has a relatively low ion strength, such as for example a solution of sucrose. Also, said vehicle may contain any solvent, aqueous or partially aqueous liquid such as non-pyrogenic sterile water. The pH of the formulation is also adjusted and buffered in order to meet in vivo requirements for use. The formulation may also include a diluent, an adjuvant or an excipient that are pharmaceutically acceptable, or solubilisation, stabilisation, conservation agents. For administration by injection a formulation in aqueous, non-aqueous or isotonic solution is preferred. It may be in single or multi-dose form, in liquid or dry form (powder, lyophilisate . . . ) able to be made up extemporaneously using an appropriate diluent. According to a particular embodiment of the invention, this composition may also contain pharmaceutically acceptable quantities of a prodrug able to be converted to a cytotoxic molecule by a polypeptide having at least cytotoxic activity. Such prodrug may be chosen in particular from the group consisting of acyclovir or ganciclovir (GCV), cyclophosphophamide, 6-methylpurine deoxyribonucloside, 6-thioxanthine, cytosine or one of its derivatives or uracil or one of its derivatives. In addition, when said prodrug is 5-fluorocytosine (5FC) or 5-fluorouracil (5-FU), said combination product may also contain one or more substances which potentialise the cytotoxic effect of 5-FU. Drugs in particular may be cited which inhibit enzymes of the de novo biosynthesis pathway of pyrimidins (for example those cited below), drugs such As Leuacovorin (Waxman et al., 1982, Eur. J. Cancer Clin. Oncol. 18, 685-692) which in the presence of the metabolism product or 5-FU (5-FdUMP) increase the inhibition of thymidylate synthase leading to reduction of the dTMP pool needed for replication, and finally drugs such as methotrexate (Cadman et al., 1979, Science 250, 1135-1137) which by inhibiting dihydrofolate reductase and raising the PRPP incorporation pool (phosphoribosylpyrophosphate) cause an increase of 5-FU in cell RNA.

Similarly, the composition to be administered may also contain a substance chosen from the group comprising for example chloroquin, protic compounds such as propylene glycol, polyethylene glycol, glycerol, ethanol, 1-methyl L-2pyrrolidone and derivatives thereof, aprotic compounds such as for example dimethylsulfoxide (DMSO), diethylsulfoxide, di-n-propylsulfoxide, dimethylsultone, sulfolane, dimethylformamide, dimethylacetamide, tetramethylurea, acetonitrile or their derivatives (see EP 890 362), cytokines, especially interleukin-10 (IL-10) (WO 9956784), hyaluronidase (WO 98/53853) and the inhibitors of nucleases (WO 9956784) such as actin G for example. In another embodiment of the invention, this substance may be a salt and preferably a cationic salt such as magnesium (MG²⁺) for example (EP 998945° and/or lithium (Li⁺). In this case, the quantity of ionic substance in the nucleic acid complex of the invention varies advantageously between 0.1 mM and approximately 10 mM.

Evidently numerous modifications may be made to the invention while remaining within its scope.

For example it is possible to use a device of the invention comprising a single catheter or two completely separate catheters, one to enter the wall and the other to administer the composition, even though this is clinically less advantageous.

It is also possible to apply the invention to ducts other than blood vessels, for example the invention may be given application in urology and gastroenterology.

Finally, the means for entering the wall could be not only mechanical. They could for example use laser sources, chemical or enzymatic means. More particularly it would be possible to use enzymes able to digest the extracellular matrix such as collagenase or hyaluronidase. Hydrolysis of collagen and hyaluronic; acid by these enzymes generates disorganisation of the extracellular matrix and facilitates access of the composition to the target cells.

Other characteristics and advantages of the invention will be seen in the following description of two preferred embodiments of the invention given as non-restrictive examples of illustration.

### EXAMPLE 1

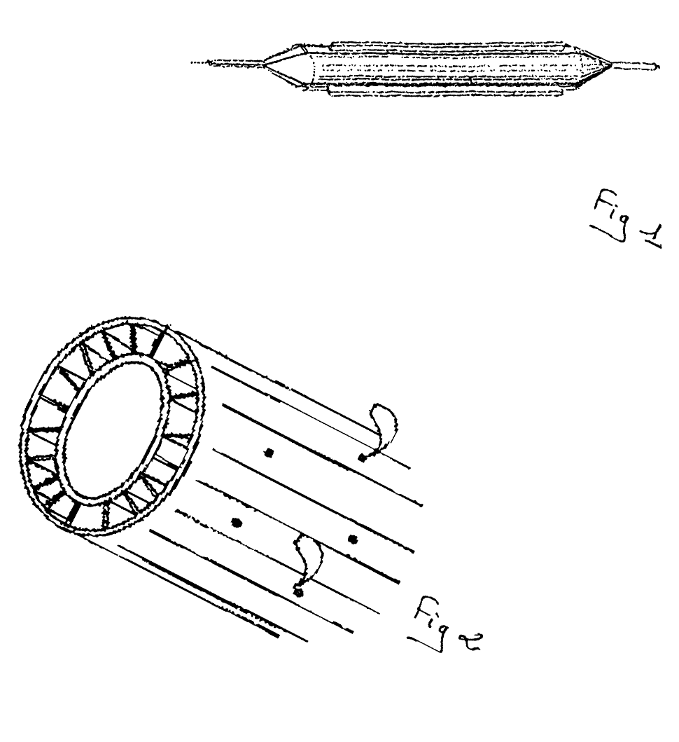

FIGS. 1 and 2 show two commercially available catheters which, when combined, make it possible to implement the method of the invention.

During a first step, the <<cutting balloons>> (FIG. 1, Interventional Technologies, U.S. Pat. No. 5,797,935) is passed forwards in the artery to the site obstructed by intra-stent restenosis. The inflatable chamber is then expanded to compress the restenosis and restore an acceptable arterial diameter. The <<cutting balloon™>> catheter, on the surface of the inflatable chamber, has three or four microsurgical razor blades. These blades are designed so that dilatation of the artery is less traumatic by making microfractures in the wall and reducing the forces exerted on the artery. When the inflatable chamber is dilated the razor blades extend radially and make incisions in the restenotic tissue.

The <<cutting halloon™>> catheter is then withdrawn and replaced by the <<Remedy balloon™>> (FIG. 2, Boston Scientific/SCIMED, U.S. Pat. No. 5,792,105) which is inserted at the dilated site of the artery. The inflatable chamber of this catheter is expanded and applies channels against the blind openings made in the artery wall by the <<cutting balloon™>>, one surface of these channels containing a wall with outer openings The composition is then dispensed via the channels and placed in contact with the arterial wall cells via the openings.

The two catheters used are given by way of a non-limitative example. In particular the <<expandable and compressible atherectomy catheter>> (U.S. Pat. No. 5,556,408), the <<universal dilator with reciprocal incisor>> (U.S. Pat. No. 5,556,405), the <<angioplasty balloon with light incisor>> (U.S. Pat. No. 5,624,433), the <<improved vascular incisor/dilator>> (U.S. Pat. No. 5,649,944), the <<device and method for transecting a coronary artery>> (U.S. Pat. No. 5,713,913) may be used in replacement of the <<cutting balloon™>>. The <<intiltrator>> (Interventional Technologies), <<Crescendo™>> (Cordis), <<InfusaSleeve™>> (LocalMed), <<Dispatch catheter™>> (Boston Scientific/SCIMED), <<Hydrogol-coated balloon catheter™>> (Boston Scientific/SCIMED) catheters may be used in replacement of the <<Remedy balloon™>>.

### EXAMPLE 2

FIGS. 3 to 10 show the different implementation steps of the method of the invention using a first embodiment of the catheter of the invention.

Catheter 2 has a distal end intended for insertion in the duct to be treated. This end comprises an inner tool 4 containing a balloon 6 of generally elongated cylindrical shape, rounded at its two axial ends. The balloon 6 is mounted on a tube 8 passing through it from end to end along its axis. A distal tip 10 of the tube emerges from the distal end of the balloon. In known manner in respect of catheters, tube 8 is hollow and is in fluid communication with the inside of the balloon. In this manner, the balloon can be inflated by supply of air through the tube from the proximal end of the catheter, not shown. Inflation of the balloon causes its radial expansion relative to the axis of the catheter.

Balloon 6 carries parts 16 on its wall, projecting beyond the outer surface, able to enter the inner surface 12 of a wall of a duct in the human body, such as an artery 14. The parts in this case are perforating parts shaped into a point, formed of crystals for example.

The distal end of the catheter also comprises an outer tool 20. This tool is termed as <<outer>> since it is intended to extend around the inner tool 4. But it is evidently intended to extend into duct 14, like the other tool. The outer tool 20 comprises a cuff 22 having a soft wall and also of general elongated cylindrical shape. This wall 22 is hollow in its centre. It is open at its distal end and has a closed proximal end of rounded shape.

This wall 22, arranged in its thickness, has long, rectilinear channels which extend parallel to the axis of the catheter. These channels have a transverse profile (in a plane perpendicular to the axis) that is generally <<U>> shaped, the bottom of the channel corresponding Lo the base of the <<U>> extending along the axis side.

As can be seen in particular in FIG. 9, the cuff 22 is connected to a tube 26 via its proximal end. The tube 8 of the inner tool slides within tube 26 of the outer tube.

The flexible cuff is extensible radially so that it can increase its diameter.

Each channel 24 is in fluid communication with the proximal end of the catheter via a distribution chamber 28 and via tube 26 to permit the supply to each channel of a liquid composition to administer to the wall of the vessel.

At the proximal end of the catheter of the invention, means are provided for actuating and controlling the distal end, and fluid injection means. This proximal end extends outside the patient's body and is operated by staff performing the procedure.

The catheter which has just been described is used in the following manner to implement the method of the invention.

It is assumed that the duct 14 to be treated is a human coronary artery. The section to be treated had a plaque of atheroma which was treated by expansion by means of a conventional balloon catheter followed by the implanting of a mesh stent 30 of a type known in itself and whose outline can be seen in FIGS. 3 to 10. After stent implantation, excessive healing 32 of the treated section occurred, reducing the inner diameter of the artery and narrowing the available opening for blood flow. The method of the invention aims at combating this excessive scar tissue. It is intended to treat restenosis by preventing re-restenosis.

With reference to FIG. 3, the distal end 2 of the catheter is passed through the artery to face the section to be treated. The inner tool 4 with the balloon deflated extends within the outer tool 20, coaxially to it.

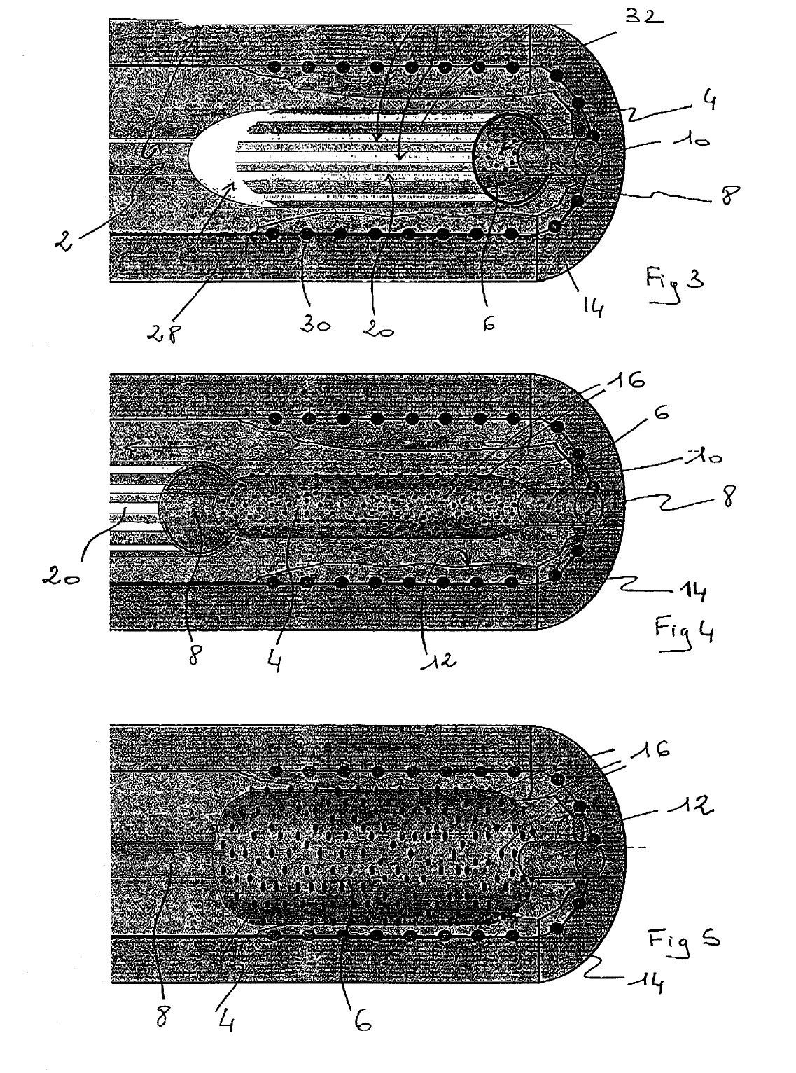

With reference to FIG. 4, once the distal end is placed opposite the section, the outer tool 20 is made to slide backwards to expose the inner tool 4.

As shown in FIG. 5, the balloon 6 is then inflated to increase its diameter so that the inner diameter of the artery is restored to acceptable size and the perforating parts 16 can penetrate the inner surface of the artery. These parts make radial blind openings 36 in the thickness of the artery wall starting from its inner surface. These openings therefore extend towards the core of the wall. These openings 36 are largely magnified in FIG. 6. They are evidently smaller and are greater in number than shown in the figure.

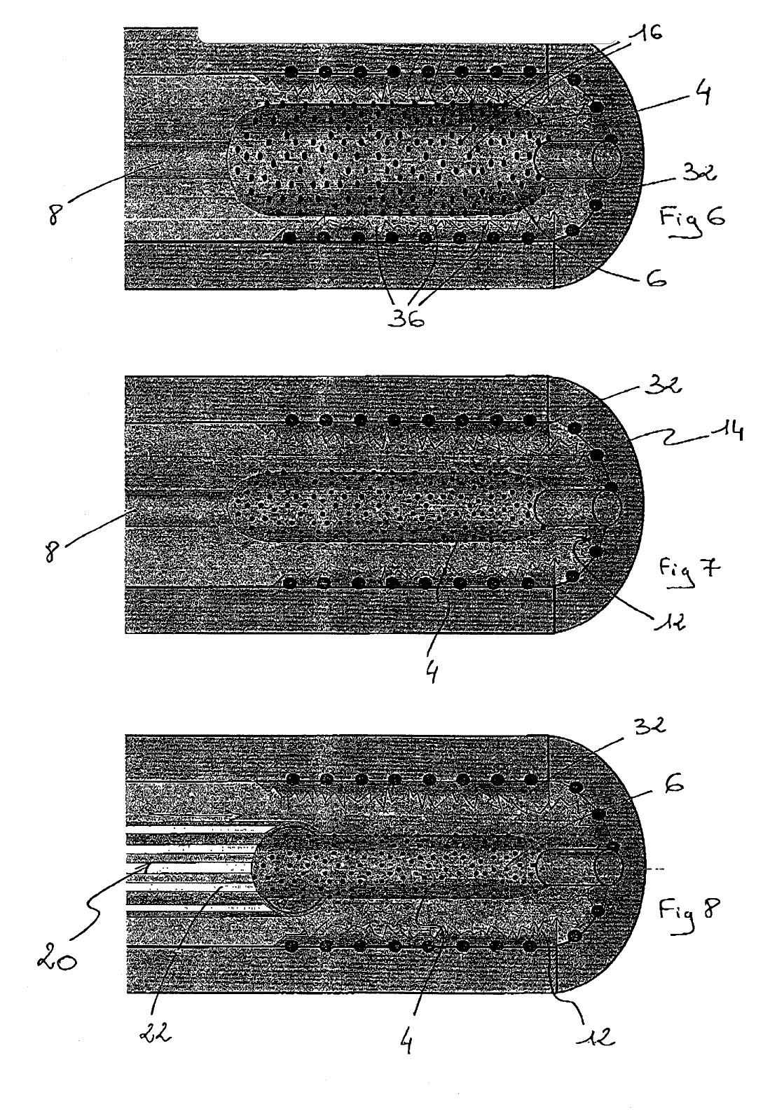

With reference to FIG. 7, the balloon 6 is then deflated to reduce its diameter.

The outer tool 20 is then caused to slide forwards in axial direction so that it surrounds the inner tool as shown in FIG. 8.

Once the outer tool is in place, balloon 6 is again inflated which causes expansion of the delivery cuff 22 as shown in FIG. 9, the channels being compressed against the inner surface of the artery which therefore closes the open surface of each channel. The composition to be administered is then injected into cuff 22. This composition circulates inside channels 24 and diffuses into all the blind openings 36 and against the inner surface of the artery. This inflation and administration step lasts a very short instant, bearing in mind that the flow of blood in the artery must not be interrupted too long.

Immediately afterwards, balloon 6 is deflated to retract cuff 22. The catheter is then removed as shown in FIG. 10.

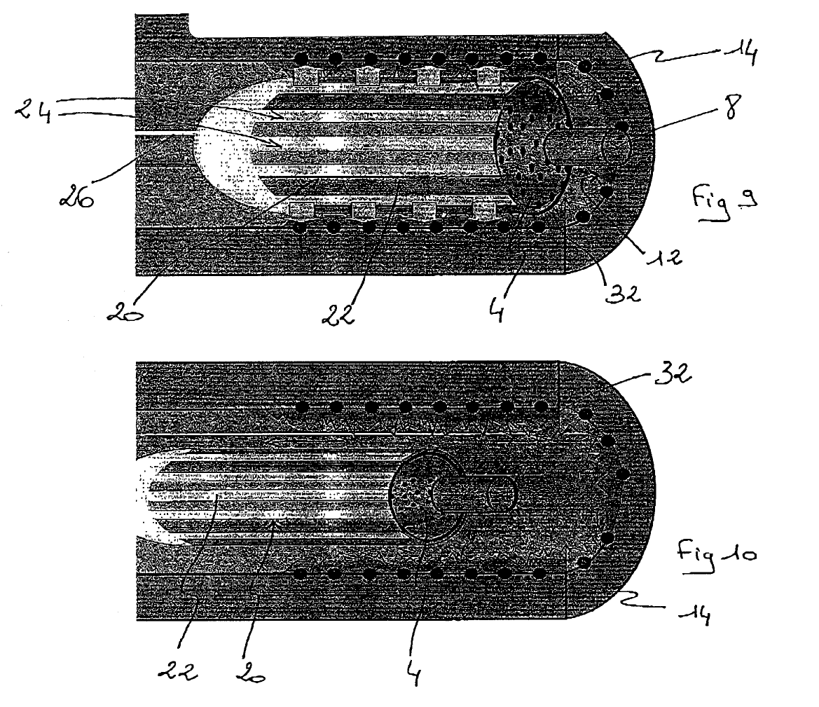

It is explained below which types of compositions can be administered using this method. A second embodiment of the catheter will now be described with reference to FIGS. 11 to 20.

### EXAMPLE 3

FIGS. 11 and 12 show the arms and the balloon in the deflated and inflated state respectively, of a catheter according to a second embodiment of the invention,

FIG. 13 is a more detailed view of the arms in FIG. 11.

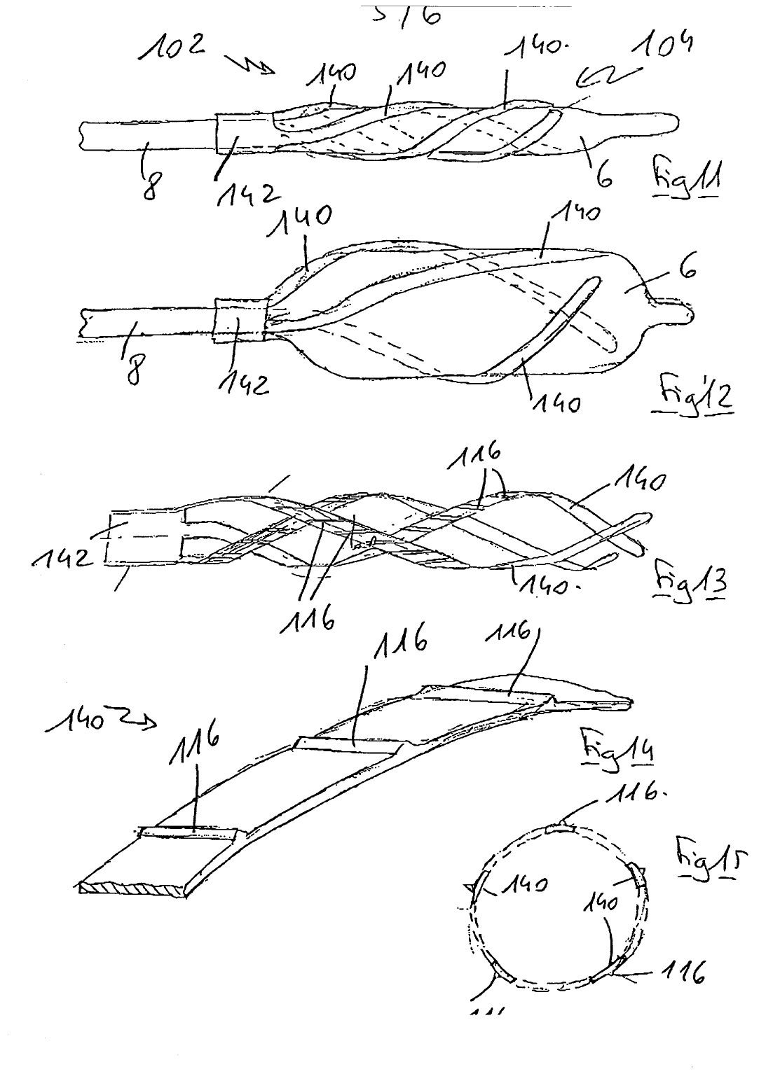

FIG. 14 is a perspective view of a section of the arm of the catheter in FIG. 13.

FIG. 15 is a cross section view of the arm assembly in FIG. 13.

FIGS. 16 to 20 illustrate implementation steps of the method of the invention using the catheter in FIGS. 11 to 15.

In this embodiment, the numerical references of similar parts are increased by 100.

The inner tool 104 shown in FIG. 11 also comprises a balloon 6 mounted on a tube 8 for its inflation. The inner tool also comprises arms 140, here three in number, carrying cutting parts. The arms are connected via their proximal end to a common cylindrical support 142 fixed to the tube. Each arm has an elongated spiral shape around the axis of the catheter, around the balloon. The three arms are evenly distributed around the axis. The three arms 140 are made in a material that is elastically flexible. They are at rest when the balloon is deflated as in FIG. 11. When the balloon is inflated, as in FIG. 12, the three arms open out elastically under the influence of the balloon. They maintain their spiral shape but the radius of the spiral becomes greater. Each arm has a local flat shape the thickness of the arm extending in a direction radial to the axis. Each arm 7 carries cutting parts on its outer surface that are here formed of sharp ridges 116 which project upwards above the outer surface. Each ridge 116 is of long rectilinear shape and extends from one side to the other of the arm edges. Here the ridges are oriented parallel to the axis of the catheter. All the ridges are therefore parallel to one another and extend from front to back. FIG. 15 shows the arrangement of the ridges and arms for a catheter comprising five arms.

With reference to FIGS. 16 to 20, the outer tool 120 of the catheter also comprises a cuff which is hollow in its centre to house the inner tool 104. The soft wall is radially expandable and hollow according to its thickness. The inner and outer surfaces of the wall are continuous but the outer surface has openings 124 to administer the composition.

The method of the invention is implemented using this catheter as follows.

It is assumed that the medical context is the same as for the first embodiment.

The inner tool 104 initially being located inside the outer tool 120, the distal end of the catheter is inserted to face the section to be treated, as shown in FIG. 16.

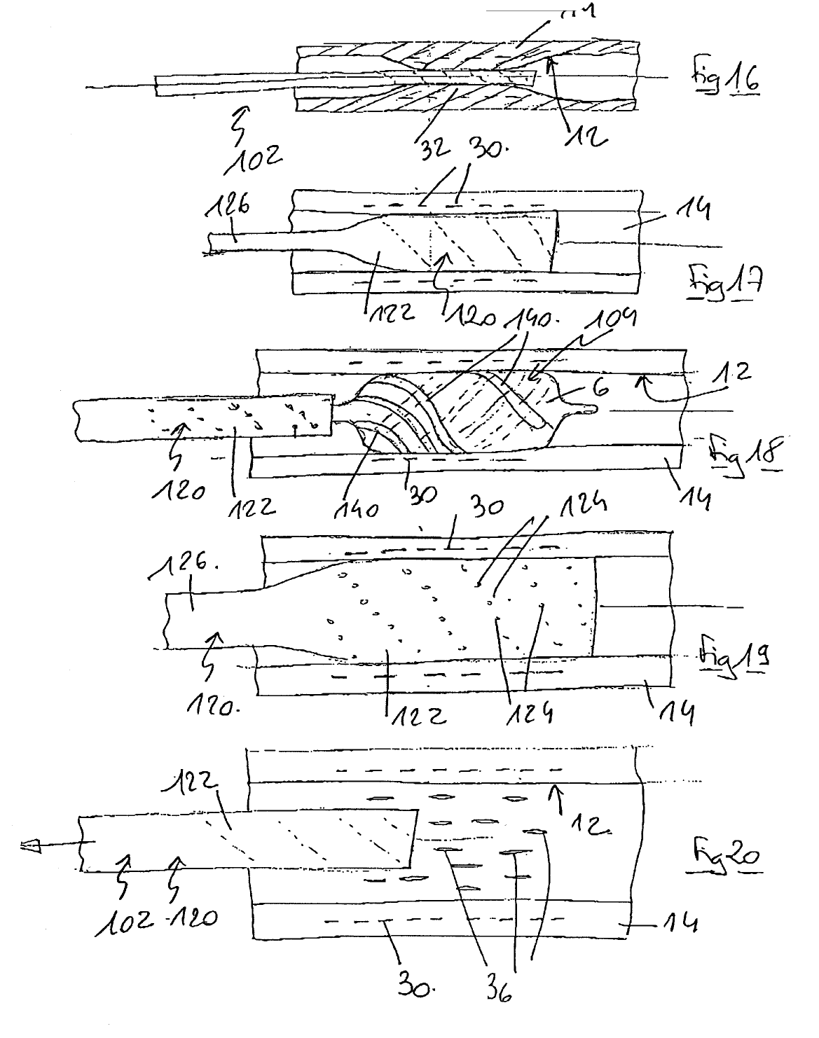

With reference to FIG. 17, balloon 6 is then inflated to expand the catheter assembly radially, in particular outer tool 120 which increases the original inner diameter of the artery.

The balloon remaining inflated, the outer tool 120 is caused to slide backwards in axial direction to place the arms 140 directly opposite the artery as shown in FIG. 18.

The balloon is then further inflated to further increase its diameter so that the sharp ridges 116 enter the arterial wall from its inner surface making blind openings 36 in the wall that follow the longitudinal direction of the artery having regard to tho orientation of the sharp ridges. The openings here are in the form of incisions that are roughly illustrated in FIG. 20. The orientation of these incisions parallel to the longitudinal direction of the artery facilitates administration of the composition.

Subsequently, with reference to FIG. 19, the balloon is partly deflated and the outer tool is caused to slide forwards over it in axial direction.

With reference to FIG. 19, the balloon is again inflated and the composition to be administered is injected into the outer tool. This composition fills the thickness of the cuff walls then escapes through openings 124 to come into contact with the inner surface of the artery and the blind openings. The balloon is then deflated and the catheter is removed as shown in FIG. 20. As in the first embodiment, the composition administering step is of very short duration.

What is claimed is:

1. Device (2; 102) for administering a composition in a wall of a duct (14) of a human or animal body, which comprises means (4; 104) to enter an inner surface (12) of the duct wall to make blind openings (36) in a thickness of the wall, and dispensing means (20; 120) to place the composition in contact with the openings.

2. Device according to claim 1, wherein the entry means (4; 104) comprise cutting parts (116) or perforating parts (16).

3. Device according to claim 1, wherein the entry means (4; 104) are radially expandable relative to an axial direction of the device.

4. Device according to any claim 1, wherein the entry means (4; 101) are associated with an inflatable chamber (6; 106).

5. Device according to claim 4, wherein the cutting or perforating means (16) are carried by a wall of the inflatable chamber (6).

6. Device according to claim 2, wherein the entry means comprise arms (140) carrying the cutting or perforating parts (116).

7. Device according to claim 6, wherein the arms (140) are associated with a tube on which an inflatable chamber (8) is mounted.

8. Device according to claim 1, wherein the dispenser means (120) are radially extensible relative to an axial direction of the device.

9. Device according to claim 1, wherein the dispenser means (20) have channels (24) able to receive the composition, the channels being open in a direction opposite to an axis of the device or closed by a wall containing openings.

10. Device according to claim 1, wherein the dispenser means (20; 120) comprise a wall having outer openings (124).

11. Device according to claim 1, wherein the dispenser means (20; 120) surround the entry means (4; 104).

12. Device according to claim 1, wherein the dispenser means (20; 120) are adopted to slide in relation to the entry means (4; 104) along an axial direction of the device.

13. Device according to claim 1, wherein the balloon (4; 104) expands the dispenser means (20; 120) radial fashion.

14. Device according to claim 1, adopted to administer a composition in the wall of a blood vessel, artery (14), or an artery carrying a stent (30).

15. Device according to claim 1, comprising a catheter.

16. Device (2; 102) for administering a composition in a wall of a duct (14) of a human or animal body, which comprises means (4; 104) to enter an inner surface of the duct wall to make blind openings (36) in the thickness of the wall, said means carrying cutting parts (116) or perforating parts (16) and being expandable radially relative to an axis of the device, the device including dispenser means (20; 120) to place the composition in contact with the openings, the dispenser means being radially expandable and adopted to surround the entry means (4; 104).
